(19) United States
(12) Patent Application Publication
(10) Pub. No.: US 2016/0317086 A1
(43) Pub. Date: Nov. 3, 2016

(54) QUICK RELEASE SYSTEM FOR WEARABLE ELECTRONIC DEVICE

(71) Applicant: OTTER PRODUCTS, LLC, Fort Collins, CO (US)

(72) Inventors: W. TRAVIS SMITH, FORT COLLINS, CO (US); CORY R. BLOOR, FORT COLLINS, CO (US)

(21) Appl. No.: 15/141,462

(22) Filed: Apr. 28, 2016

Related U.S. Application Data
(60) Provisional application No. 62/156,130, filed on May 1, 2015.

(51) Int. Cl. A61B 5/00; H02J 7/00

(52) U.S. Cl. CPC A61B 5/681; H02J 7/0044; H02J 7/0052

(57) ABSTRACT

A releasable holder for a wearable electronic device, the releasable holder comprising a shell, a hinged clasp, and a release switch. The shell is configured to engage a first slot of the wearable electronic device against a first wall of the shell. The hinged clasp is attached to a second wall of the shell and the hinged clasp is configured to engage a second slot of the wearable electronic device, the shell and hinged clasp together retaining the wearable electronic device within the shell of the releasable holder. A release switch is attached to the second wall of the shell and configured to disengage the clasp from the second slot of the wearable electronic device, releasing the wearable electronic device from the shell.

### CROSS-REFERENCE TO RELATED APPLICATIONS

This application claims priority to U.S. Provisional Patent Application No. 62/156,130 filed May 1, 2015, all of which is expressly incorporated by reference in its entirety.

### FIELD

This disclosure relates generally to cases, covers, shells, enclosures, and bands for electronic devices. More specifically, the instant application relates to cases, covers, shells, enclosures, and bands for wearable electronic devices.

### BACKGROUND

Portable electronic devices are commonly used for communication, entertainment, and/or information purposes. Portable electronic devices include devices such as smartphones, cellular phones, mobile communication devices, computers, portable computing devices, mobile computing devices, tablet computers, cameras, video players, audio players, electronic media readers, two-way radios, global positioning satellite (GPS) devices, and/or other types of electronic computing or communication devices, including combinations thereof. Recent advances in electronics and miniaturization have allowed some or all of the functions of these devices to be implemented in even smaller electronic devices that can be worn on a user's body. Wearable electronic devices can include watches, smartwatches, fitness monitors, activity monitors, biometric monitors, medical monitors, functional jewelry, and interactive implementations thereof.

In some situations, a wearable electronic device can be a standalone device, in the sense that it performs a variety of electronic functions without relying on any other devices. In other situations, a wearable electronic device can operate in conjunction with another electronic device that is carried by or near the user. In one specific example, a smartwatch (e.g. the APPLE WATCH®, PEBBLE®, SAMSUNG® GEAR, etc.) worn by a user provides a number of functions and can communicate with or through a smartphone that is separately carried by the user or is placed somewhere near the user. In this example, the smartwatch makes certain features or functions more readily available to a user without the user having to take the smartphone out of a pocket or bag and/or without having to activate the screen of the smartphone. However, in other situations, the smartwatch can operate as a standalone device without relying on a nearby smartphone. While many of the examples herein are described with respect to a smartwatch, the apparatuses and techniques disclosed herein are also applicable to other types of electronic devices including fitness monitors, activity monitors, biometric monitors, medical monitors, functional jewelry, and the like. While described as a “watch,” a smartwatch can provide many functions other than providing the time including, providing indications of received email messages or text messages, providing calendar information, providing appointment information or reminders, receiving inputs from the user, displaying weather information, capturing biometric information, and/or interactive implementations thereof.

As with other types of electronic devices, wearable electronic devices are subject to damage from shock, vibration, impact, external forces, sharp objects, water, dirt, dust, snow, rain, sweat, chemicals, and/or other damaging elements. In some situations, wearable electronic devices can be even more susceptible to some of these damaging forces or elements because they are worn on the body and/or because of where they are worn on the body. Damaging forces can cause the wearable electronic device to no longer be operable or can simply damage the physical appearance of the wearable electronic device. In some instances, wearable electronic devices can have bands, chains, and/or other attachment mechanisms that are removable or changeable such that a user can be able to change or add their own band, chain, and/or other attachment mechanism. Improved protection and/or more durable bands for these types of wearable electronic devices are needed.

Wearable electronic devices can also have multiple connection and release mechanisms built into the housing of the device for attaching wristbands or other items. However, such connection mechanisms can be small in size and have features that limit the probability of accidental device release (e.g. recessed buttons). Such connection and release mechanisms, while secure, can limit the speed and ease with which a user can insert and remove the wearable electronic device from a band.

It is therefore desirable that a wearable electronic device can be easily and quickly moved between different wearable bands, attached to a charging station or stand, or otherwise mounted. It is also desirable that the wearable electronic device can be removed from a wearable band for charging or other use, without also removing the wearable band from the body.

### SUMMARY

A quick release mechanism for a wearable electronic device can be integrated with a shell and a wearable band, with a charging station, with a bike mount, a shell having a clip, or a shell attached to some other attachment mechanism or element (e.g. suction cup, magnet, etc.). The quick release mechanism can include a moveable clasp that allows the wearable electronic device to be pressed into a shell, stand, or charging station and be retained. The rapid connection and release mechanism can also include a button or release switch that is pressed or activated to release the clasp and allow the wearable electronic device to be removed from the shell, stand, or charging station.

The summary of the invention described above is non-limiting and other features and advantages of the invention will be apparent from the following detailed description of the invention, and from the claims.

### BRIEF DESCRIPTION OF THE FIGURES

Embodiments of the present technology are described and explained using the accompanying drawings summarized below.

FIG. 1A illustrates a front plan view of an exemplary watch band and protective shell that includes a quick release mechanism for a wearable electronic device.

FIG. 1B shows a perspective view of the shell of FIG. 1A.

FIG. 1C illustrates a side view of a user pressing the release button of the shell and the wearable electronic device partially engaged with the shell.

FIG. 1D shows a perspective view of a wearable electronic device partially engaged with a shell.

FIG. 2 illustrates a perspective view of a wearable electronic device charging stand with a quick connection and release mechanism.

FIG. 3 shows a view of a handlebar mount for a wearable electronic device that includes a quick connection and release mechanism.

FIG. 4A illustrates a rear plan view of a wearable electronic device shell that includes a reversible clip arm.

FIG. 4B shows a front plan view of the wearable electronic device shell of FIG. 4A.

FIG. 4C illustrates a front perspective view of the wearable electronic device shell of FIGS. 4A and 4B.

FIG. 5A shows a rear perspective view of a wearable electronic device shell with a reversible arm.

FIG. 5B shows a front perspective view of the wearable electronic device shell of FIG. 5A with the reversible clip arm over the front of the shell.

FIG. 5C shows a view of a wearable electronic device shell attached to the exterior of a user's waistband, with the front face of the wearable electronic device facing outward.

FIG. 5D illustrates a view of a wearable electronic device shell attached to the inside of a user's waistband with the rear portion of the smartwatch shell facing the skin of the user.

### DETAILED DESCRIPTION OF THE DISCLOSURE

The present disclosure is directed to shells, encasements, and charging devices for wearable electronic devices that include a rapid connection and release mechanism. The wearable electronic device can be quickly and easily inserted into the shell, and secured and protected until the connection and release mechanism is activated and the electronic device is removed.

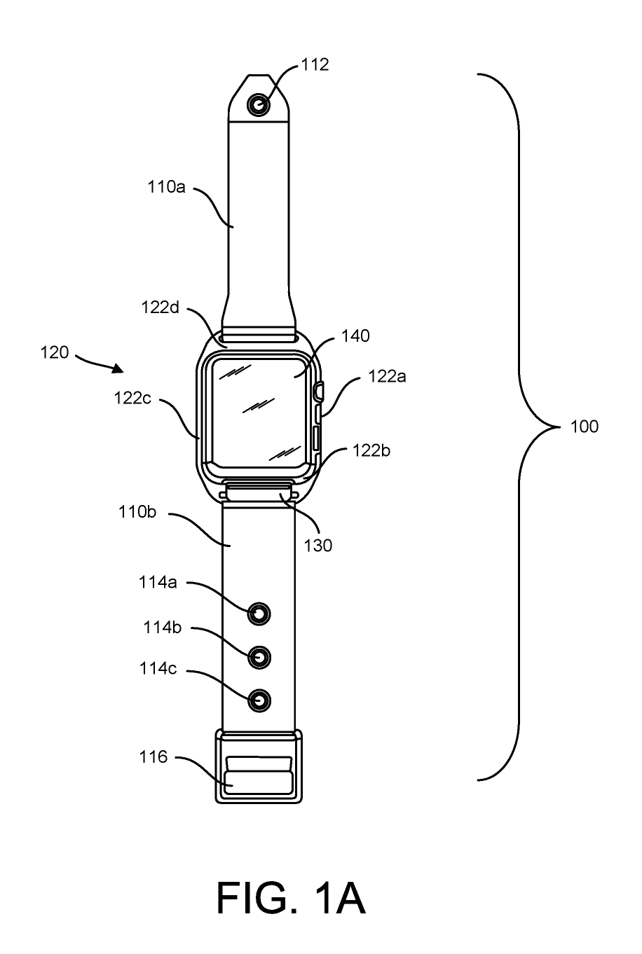

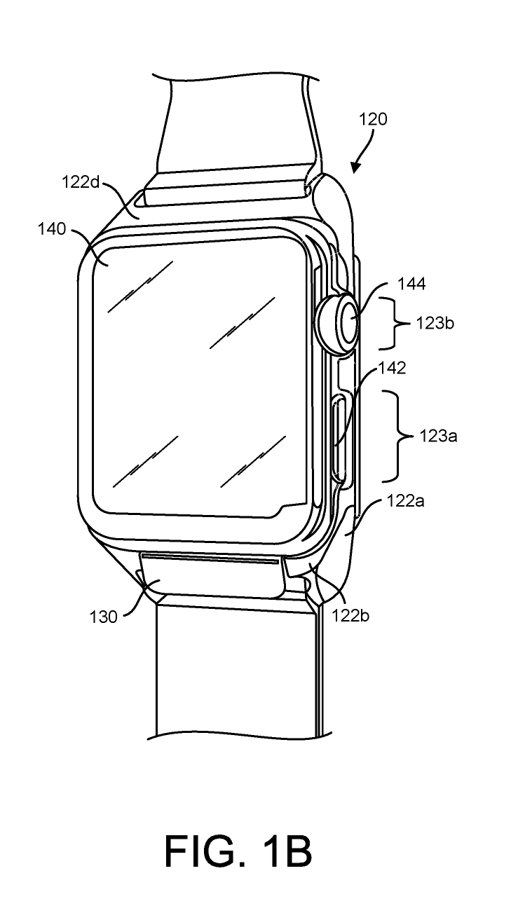

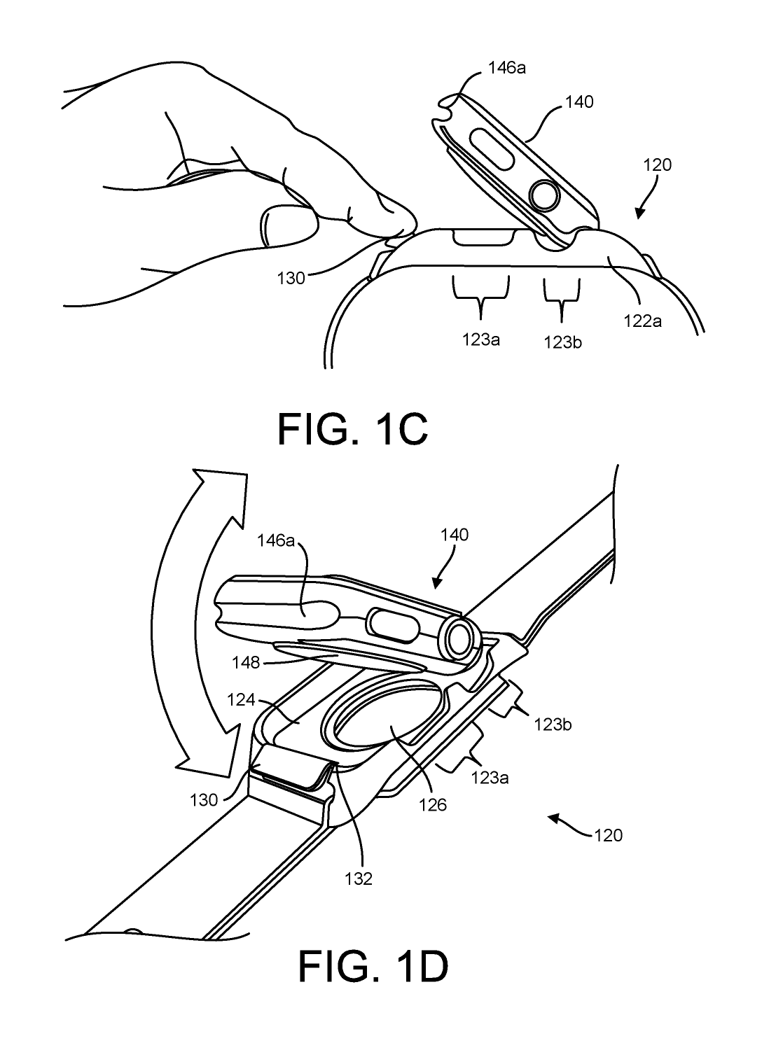

FIGS. 1A-1D illustrate various views of a wearable electronic device in a protective banded holder with quick release connection mechanism. Banded holder can also be referred to as banded shell, banded encasement, or a protective banded shell. Banded holder 100 includes wristbands 110a and 110b attached to proximal and distal ends of shell 120. Although wristbands are depicted, bands configured to wrap around other body parts, such as an arm, leg, neck, head, or other body can be used. Bands can also be configured to wrap around other objects as well, including bars, handlebars, posts, or the like. Wristbands 110a and 110b include snap protrusion 112 and reciprocal snap holes or apertures 114a, 114b, and 114c, configured to receive snap protrusion 112. In some embodiments, wristbands 110a and 110b can be integrally formed with shell 120, or can be a single band that is attached to shell 120 (e.g. through slots on the rear surface of shell 120). Wristband 110a can be inserted through adjustment loop 116 and snap protrusion 112 is snapped into one of snap holes 114a-114c. In some embodiments, wristband 110a can include more than one snap protrusion. Other fasteners can be utilized on wristband 110a, such as a buckle, hook and loop fasteners, buttons, magnets, Velcro, and folding clasps.

Shell 120 has walls 122a, 122b, 122c, and 122d, defining cavity 124 (partially visible in FIG. 1D) to hold and retain wearable electronic device 140. In some instances, shell walls 122a-122d can be configured to extend around to the front surface of installed wearable electronic device to further retain wearable electronic device 140. FIG. 1B illustrates a perspective view of protective banded holder 100, with wall 122a having cutouts 123a and 123b to allow access to functional features of wearable electronic device 140, such as button 142 and rotary knob 144 of wearable electronic device 140. In some embodiments, walls 122a and 122c of shell 120 can have holes to allow access to button 142 and rotary knob 144 of wearable electronic device 140. In other embodiments, shell 120 can include a button actuator overmolded onto holes or cutouts of shell 120, to allow button 142 of wearable electronic device 140 to be activated through wall 122a.

Shell 120 includes a connection mechanism having release switch 130 and clasp 132. Release switch 130 can be pressed to unlatch the connection mechanism and release wearable electronic device 140 from shell 120. Release switch can also referred to as an actuator, quick release actuator, activator, or button. FIG. 10 shows a side view of protective banded holder 100 with release switch 130 being pressed, and wearable electronic device 140 partially released from shell 120. In some implementations, wearable electronic device 140 has slots 146a and 146b (not visible) on respective proximal and distal ends. Slots can also be referred to as grooves, indents, or slotted openings. Slots 146a and 146b can be rounded and the opening of each slot can be angled downward and away from the display of wearable electronic device 140. Wearable electronic device 140 depicted in FIGS. 1A-1D is configured to be removably engaged directly with other wristbands using slots 146a and 146b as guides for the ends of wristbands to slide into slots 146a and 146b and lock in place. The distal end of shell 120 on the interior portion of wall 122d can include a rod or curved protrusion (not shown) within cavity 124 that is configured to interface with slot 146b (not visible) and aid in retaining wearable electronic device 140 inside cavity 124. Release switch 130 can be mechanically connected to clasp 132 that fits inside of slot 146a and interfaces with slot 146a to aid in retaining wearable electronic device 140 in shell 120. In some embodiments, clasp 132 can be shaped to at least partially fill slot 146a. Release switch 130 can be hinged such that it can actuate back and forth, allowing clasp 132 to enter and exit slot 146a. Release switch 130 can be tensioned (e.g. with a spring) such that release switch 130 is in an upward or raised position, can be pushed down when activated, and then returns to the upward or raised position after release switch 130 is pressed. In some embodiments, release switch 130 can be configured as a switch that is pressed inward and springs back in place to activate and move the clasp 132 into slot 146a.

FIG. 1D indicates how wearable electronic device 140 is inserted and removed from shell 120. The distal end of wearable electronic device 140 is inserted into the corresponding distal end of cavity 124 and against shell wall 122d such that slot 146b (not visible) interfaces with a rod or curved protrusion in shell 120. In some embodiments, slot 146b may not interface with a rod or curved protrusion and instead rest against or within shell wall 122d. The proximal end of wearable electronic device 140 is then lowered into the corresponding portion of cavity 124 that includes clasp 132. The proximal end of wearable electronic device 140 is then pressed downward into cavity 124. Clasp 132 is pressed outward against the wearable electronic device until slot 146a is reached, and then interfaces and engages with slot 146a to retain wearable electronic device 140. To remove wearable electronic device 140 from shell 120, release switch 130 is pressed or pushed, moving clasp 132 from slot 146a and allowing the proximal portion of wearable electronic device to be removed from cavity 124. Wearable electronic device 140 can then be angled upward allowing slot 146b (not visible) to be released from the corresponding rod or curved protrusion in the distal portion of shell 120.

Shell 120 can also include a sensor aperture or sensor opening 126, configured to allow access to sensor region 148 (e.g. a region including one or more biometric or other sensors) on the rear surface of wearable electronic device 140. The rear surface of the shell can also be referred to as the bottom surface or bottom wall. Aperture 126 can also allow access to charging of wearable electronic device 140 without removing wearable electronic device 140 from shell 120 of protective holder 120. Thus, when banded holder 100 is worn with installed wearable electronic device 140, sensor region 148 can access the skin of the underlying body part. In other instances, shell 120 does not include a back portion of cavity 124 and only includes side walls 122a, 122b, 122c, and 122d. In other instances, shell includes a variation of side walls 122a-122d, including a variation of only having side wall 122b and 122d.

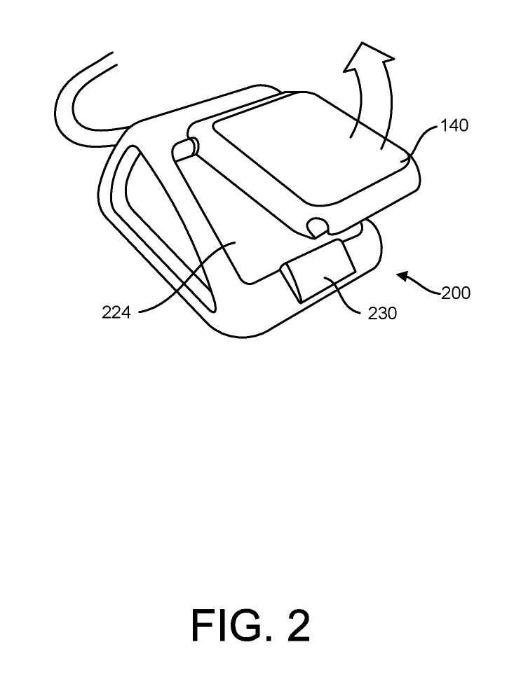

In some instances, the quick release connection mechanism and shell can be referred to as a releasable encasement or a quick release holder. In some instances, the releasable encasement can be integrated or attached to a stand or station. FIG. 2 shows charging station 200 that includes release switch 230 and associated clasp mechanism (not visible) similar to that described above. In FIG. 2, wearable electronic device 140 is shown partially released from cavity 224. As described above, the proximal end of wearable electronic device 140 is inserted first, followed by the distal end until the clasp is engaged. To release wearable electronic device 140, release switch 230 is pressed, disengaging the clasp mechanism and allowing the distal end of wearable electronic device 140 to be removed, followed by the proximal end of wearable electronic device 140. The charging station can include electronic circuitry for controlling charging of the wearable electronic device, and can include one or more of a wireless charging antenna/coil and an electrical connector for transmission of electrical power to the wearable electronic device. The electronic circuitry can also include communications circuitry for communicating data to/from the electronic device. The charging station can include a power input cable and may, for portability, include a rechargeable battery that can be used as a source of power for the wearable electronic device.

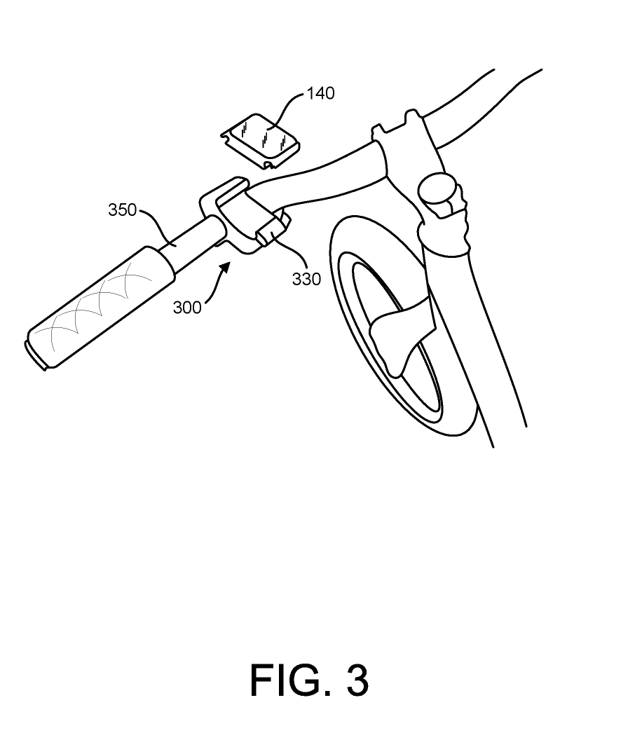

FIG. 3 illustrates bike mount shell 300 that is configured to attach to handlebar 350 of a bicycle. Bike mount shell 300 also includes a quick connection and release mechanism similar to that described above. Bike mount shell 300 can be attached to handlebar using a strap, zip ties, a rigid or semi-rigid collar that is secured with a clasp or screw, an adhesive, or the like.

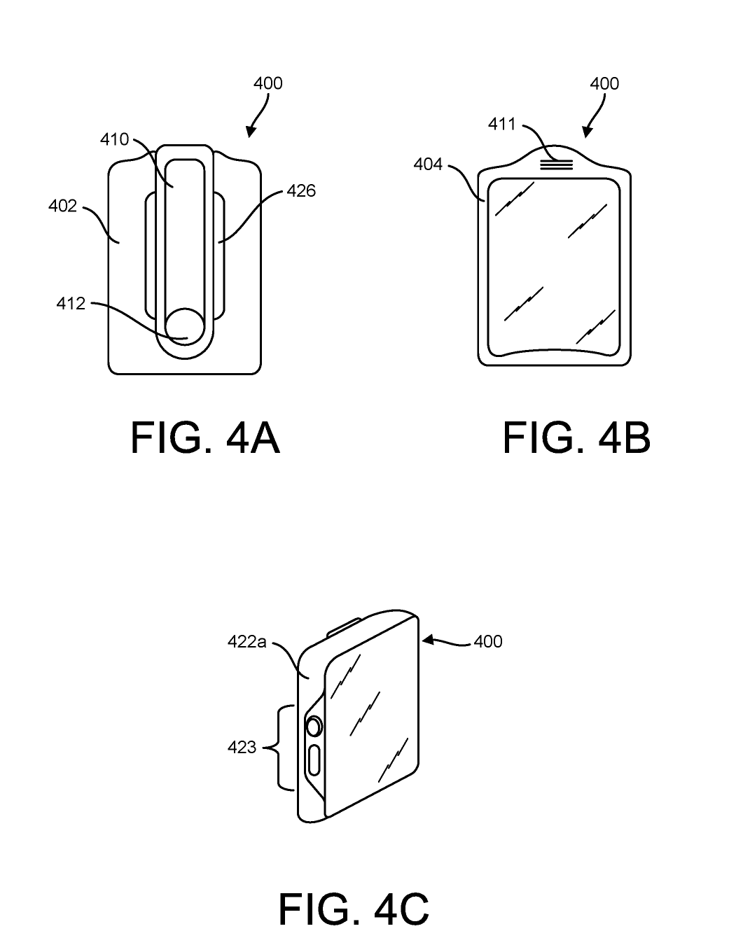

In some embodiments, a shell having a quick release mechanism such as that described above can be attached to a clip, for clipping the device to an article of clothing (e.g. waistband, headband), bag strap, or other item. FIGS. 4A-4E illustrate views of shell 400 that includes clip arm 410 proximate the rear portion of shell 400. In the depicted embodiment, clip arm 410 is connected by a hinge to rear surface 402 of shell 400, and includes a spring (not shown) that forces clip arm 410 into a closed position. Rear surface 402 of shell 400 can also be referred to as an outer surface. FIG. 4A shows a plan view of the rear surface 402 of shell 400. Shell 400 is configured such that wearable electronic device 140 can be inserted with the display facing outward, as well as inward towards inner surface of shell 400. Rear surface 402 includes aperture 426, which can or can not include a membrane that protects the display of the watch when inserted inward. FIG. 4B shows the front surface of shell 400 (clip arm 410 is not visible). Protrusion 411 provides a user with a surface to pinch the upper end of clip arm 410 and levering open clip arm 410 for attachment to a strap or piece of clothing. In some instances, clip arm 410 can include a flexible material, such as a woven or non-woven fabric. The interior portion of clip arm 410 can include a stiffener, such as metal, plastic, or other polymer. The distal end of clip arm 410 (where clip arm opens to receive a strap or portion of clothing) can include releasable attachment element 412, such as a snap, a magnet, a hook and loop fastener, and the like. FIG. 4C shows a perspective view of shell 400 with control cutout 423 in perimeter wall 422a. In some embodiments, shell 400 can reversibly attach to a rigid clip arm that can be removed and reversed in orientation. An end of clip arm 410 can be attached to shell 400 by a living hinge or by a flexible material, such that clip arm can be flipped onto the front surface of shell 400. The clip arm can include a rigid or semi-rigid material. A releasable attachment element (not visible) can attach to both the rear surface 402 and the front surface 404 of shell 400.

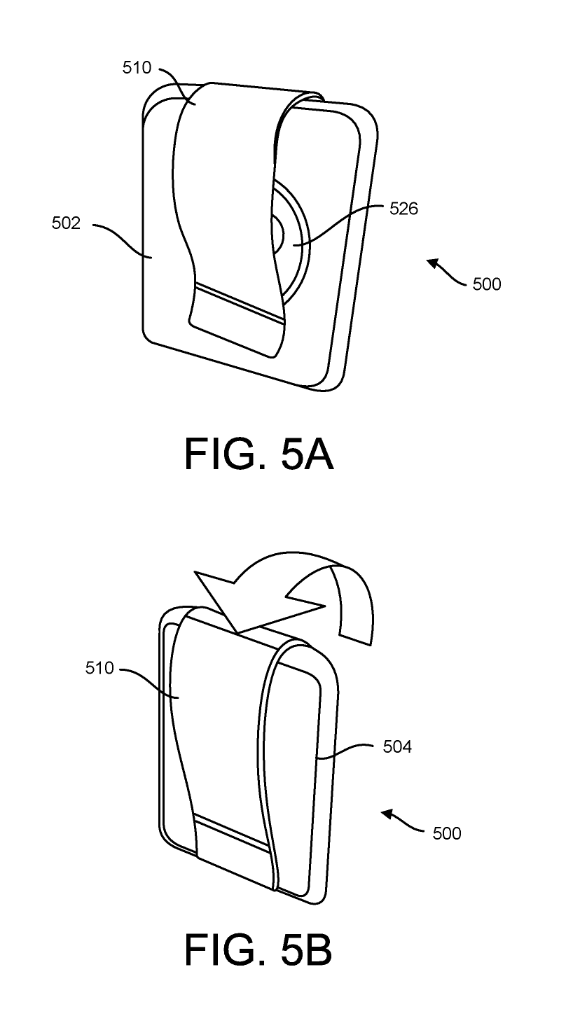

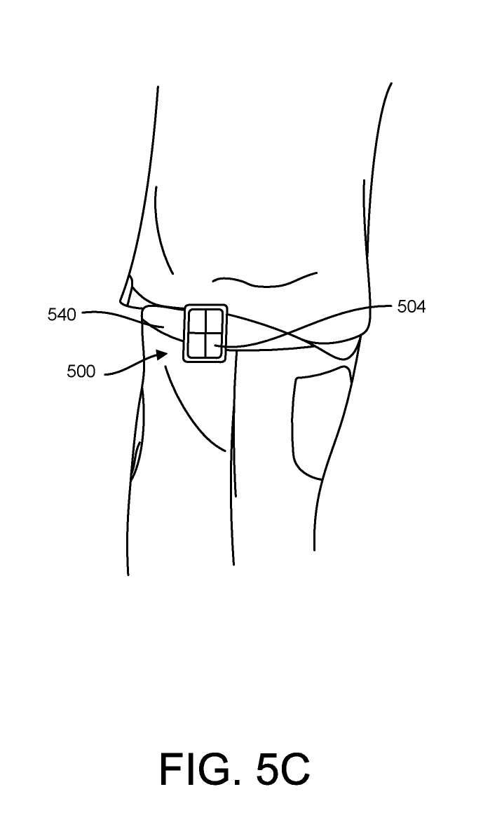

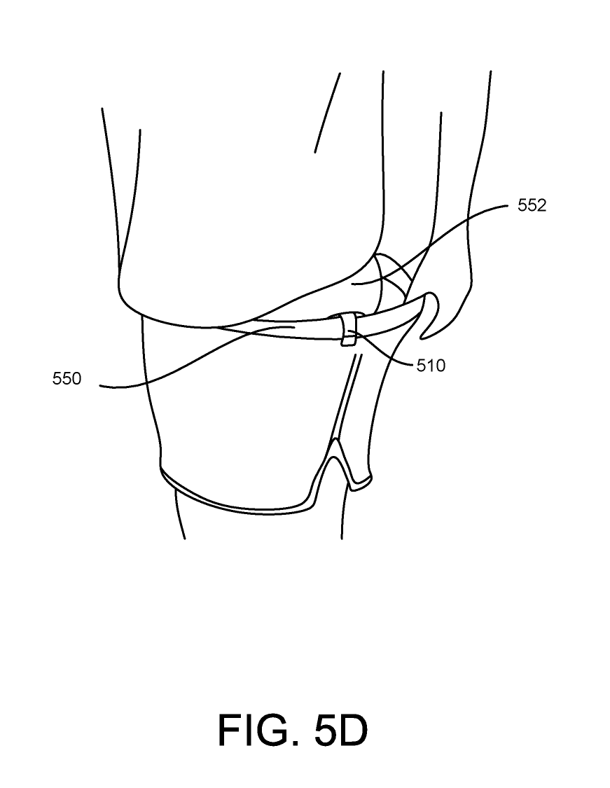

In some embodiments, a clip arm for a shell can be configure as a flexible, reversible clip arm. For example, FIG. 5A illustrates a perspective view of rear surface 502 of shell 500 that includes reversible clip arm 510. Reversible clip arm 510 is proximate rear surface 502 of shell 500, leaving the display of wearable electronic device 140 visible. FIG. 5B shows shell 500 in a different configuration, with reversible clip arm 510 moved from the rear surface 502 to front surface 504 of shell 500 (arrow indicates direction of clip arm movement). These two configurations of reversible clip arm 510 allows a wearable electronic device installed in the case to be attached to an item of clothing in two different configurations, illustrated in FIGS. 5C and 5D. For example, FIG. 5C shows shell 500 with clip arm 510 proximate the rear surface (not visible) and clipped onto a user's waistband 540, with front surface 504 of shell 500 facing outward, away from the waistband. Clip arm 510 (not visible) is between the interior of waistband 540 and the body of the user. FIG. 5D shows a view of shell 500 in alternative configuration on waistband 550. Clip arm 510 is facing outward from waistband 550. In this configuration, clip arm 510 is proximate the front surface of shell 500, separated by the fabric of waistband 550. Biometric or other sensors on the rear surface of the wearable electronic device (not visible) can thus be placed against the user's skin 552. This configuration of shell 500 can provide additional security against the shell and wearable electronic device being dislodged. If the waistband is elastic or includes a belt, the waistband can press the wearable electronic device against the user's body while minimizing the chances of the wearable electronic device being moved during exercise.

The accompanying figures can depict exemplary configurations for an apparatus of the disclosure, which is done to aid in understanding the features and functionality that can be included in the housings described herein. The apparatus is not restricted to the illustrated architectures or configurations, but can be implemented using a variety of alternative architectures and configurations. Additionally, although the apparatus is described above in terms of various exemplary embodiments and implementations, it should be understood that the various features and functionality described in one or more of the individual embodiments with which they are described, but instead can be applied, alone or in some combination, to one or more of the other embodiments of the disclosure, whether or not such embodiments are described and whether or not such features are presented as being a part of a described embodiment. Thus the breadth and scope of the present disclosure, especially in any following claims, should not be limited by any of the above-described exemplary embodiments.

The term “about” as used herein in reference to quantitative measurements, refers to the indicated value plus or minus 10%.

Terms and phrases used in this document, and variations thereof, unless otherwise expressly stated, should be construed as open ended as opposed to limiting. As examples of the foregoing: the term “including” should be read to mean “including, without limitation” or the like; the term “example” is used to provide exemplary instances of the item in discussion, not an exhaustive or limiting list thereof; and adjectives such as “conventional,” “traditional,” “standard,” “known” and terms of similar meaning should not be construed as limiting the item described to a given time period or to an item available as of a given time, but instead should be read to encompass conventional, traditional, normal, or standard technologies that can be available or known now or at any time in the future. Likewise, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although item, elements or components of the disclosure can be described or claimed in the singular, the plural is contemplated to be within the scope thereof unless limitation to the singular is explicitly stated. The presence of broadening words and phrases such as “one or more,” “at least,” “but not limited to” or other like phrases in some instances shall not be read to mean that the narrower case is intended or required in instances where such broadening phrases can be absent. Additionally, where a range is set forth, the upper and lower limits of the stated range are inclusive of all of the intermediary units therein.

The foregoing description is intended to illustrate but not to limit the scope of the disclosure, which is defined by the scope of the appended claims. Other embodiments are within the scope of the following claims.

1. A releasable holder for a wearable electronic device, the releasable holder comprising:
a shell having a first wall, a second wall, and a bottom wall, the first, second, and bottom walls configured to receive the wearable electronic device, the first wall having a protrusion configured to partially engage with a first slot of the wearable electronic device;
a hinged clasp attached to the second wall of the shell, the hinged clasp configured to at least partially engage with a second slot of the wearable electronic device and retain the wearable electronic device in the releasable holder; and
a release switch attached to one of the walls of the shell, the release switch configured to disengage the hinged clasp from the second slot of the wearable electronic device to release the wearable electronic device from the releasable holder.

2. The releasable holder of claim 1, wherein the shell comprises an opening in the bottom wall, the opening configured to allow access to a sensor of the wearable electronic device while the wearable electronic device in installed in the releasable holder.

3. The releasable holder of claim 2, wherein the sensor of the wearable electronic device is a biometric sensor.

4. The releasable holder of claim 1, further comprising a clip attached to an outer portion of the shell, the clip configured to attach to an article of clothing.

5. The releasable holder of claim 1, further comprising a wristband attached to the shell.

6. The releasable holder of claim 1, wherein the shell is attached to a handlebar.

7. A releasable encasement for a wearable electronic device, the releasable encasement comprising:
a shell configured to retain the wearable electronic device;
a movable clasp attached to a wall of the shell, the movable clasp configured to engage with a portion of the wearable electronic device to retain the wearable electronic device within the shell of the releasable encasement; and
an actuator attached to the movable clasp, the actuator configured to allow the movable clasp to engage the wearable electronic device when the wearable electronic device is inserted into the shell of the releasable encasement to removably retain the wearable electronic device in the shell, the actuator further configured to disengage the movable clasp from the portion of the wearable electronic device to remove the wearable electronic device from the shell of the releasable encasement.

8. The releasable encasement of claim 7, wherein the shell comprises a protrusion configured to partially engage with a first slot of the wearable electronic device.

9. The releasable encasement of claim 8, wherein the movable clasp is configured to engage with a portion of the wearable electronic device and partially engage with a second slot of the wearable electronic device.

10. The releasable encasement of claim 7, wherein the shell is configured to allow access to a biometric sensor of the wearable electronic device.

11. The releasable encasement of claim 7, wherein the shell is configured to allow access to a rotary knob of the wearable electronic device.

12. The releasable encasement of claim 7, wherein the shell is integrated into a charging station configured to charge the wearable electronic device.

13. The releasable encasement of claim 7, wherein the shell is configured to mount on a bicycle handlebar.

14. The releasable encasement of claim 7, wherein the shell is attached to an adjustable wristband.

15. A releasable holder for a wearable electronic device, the releasable holder comprising:
a shell for encasing at least a portion of the wearable electronic device;
a clasp for engaging and retaining the wearable electronic device in the shell; and
a quick release actuator for actuating the clasp to release the wearable electronic device from the shell.

16. The releasable holder of claim 15, wherein the shell comprises cutouts that allow access to functional features of the wearable electronic device while the wearable electronic device is in the shell.

17. The releasable holder of claim 16, wherein at least one of the functional features of the wearable electronic device is a rotary knob.

18. The releasable holder of claim 15, further comprising an adjustable wristband attached to the shell.

19. The releasable holder of claim 15, further comprising a stand attached to the shell.

20. The releasable holder of claim 19, wherein the stand is configured to hold a charging device for the wearable electronic device.
